(19) United States
(12) Patent Application Publication
(10) Pub. No.: US 2024/0339191 A1
(43) Pub. Date: Oct. 10, 2024

(54) PREDICTING OPTIMAL TREATMENT REGIMEN FOR NEOVASCULAR AGE-RELATED MACULAR DEGENERATION (NAMD) PATIENTS USING MACHINE LEARNING

(71) Applicants: Genentech, Inc., South San Francisco, CA (US); Hoffmann-La Roche Inc., Little Falls, NJ (US)

(72) Inventors: Yusuke Alexander KIKUCHI, South San Francisco, CA (US); Ales NEUBERT, Basel (CH); Carlos Quezada RUIZ, South San Francisco, CA (US); Jian DAI, South San Francisco, CA (US)

(21) Appl. No.: 18/744,107

(22) Filed: Jun. 14, 2024

Related U.S. Application Data
(63) Continuation of application No. PCT/US2022/081880, filed on Dec. 16, 2022.
(60) Provisional application No. 63/291,275, filed on Dec. 17, 2021.
(60) Provisional application No. 63/330,753, filed on Apr. 13, 2022.
(60) Provisional application No. 63/415,949, filed on Oct. 13, 2022.

(51) Int. Cl. G16H 20/17; A61B 3/00; A61B 3/10; A61B 3/12; G16H 50/30

(52) U.S. Cl. CPC G16H 20/17; A61B 3/0025; A61B 3/102; A61B 3/1225; G16H 50/30

(57) ABSTRACT

A method and system for predicting a selected treatment regimen for a subject. Baseline data for a subject diagnosed with neovascular age-related macular degeneration (nAMD) is received. A plurality of predictor inputs is formed for an outcome predictor using the baseline data and regimen data for a plurality of treatment regimens. The plurality of predictor inputs comprises a different predictor input for each of the plurality of treatment regimens. A plurality of treatment scores is generated for the plurality of treatment regimens via the set of outcome predictor using the plurality of predictor inputs. One of the plurality of treatment regimens is selected as a selected treatment regimen for the subject based on the plurality of treatment scores.

### CROSS-REFERENCE TO RELATED APPLICATIONS

This application is a continuation of International Application No. PCT/US2022/081880, filed on Dec. 16, 2022, and entitled “Predicting Optimal Treatment Regimen for Neovascular Age-Related Macular Degeneration (NAMD) using Machine Learning,” which claims priority to U.S. Provisional Patent Application No. 63/415,949, filed on Oct. 13, 2022, and entitled “Predicting Optimal Treatment Regimen for Neovascular Age-Related Macular Degeneration (NAMD) using Machine Learning,” U.S. Provisional Patent Application No. 63/330,753, filed on Apr. 13, 2022, and entitled “Predicting Optimal Treatment Regimen for Neovascular Age-Related Macular Degeneration (NAMD) using Machine Learning,” and U.S. Provisional Patent Application No. 63/291,275, filed on Dec. 17, 2021, and entitled “Predicting Optimal Treatment Regimen for Neovascular Age-Related Macular Degeneration (NAMD) using Machine Learning,” each of which is incorporated herein by reference in its entirety.

### FIELD

This description is generally directed towards predicting an optimal treatment regimen to use for subjects diagnosed with neovascular age-related macular degeneration (nAMD). More specifically, this description provides methods and systems for selecting one out of various potential treatment regimens to use for a subject with nAMD based on treatment scores for the treatment regimens generated using machine learning models.

### BACKGROUND

Age-related macular degeneration (AMD) is a disease that impacts the central area of the retina in the eye, which is referred to as the macula. AMD is a leading cause of vision loss in subjects 50 years or older. Neovascular AMD (nAMD) is one of the two advanced stages of AMD. With nAMD, new and abnormal blood vessels grow uncontrollably under the macula and may leak fluid from the blood into the retina. This type of growth may cause swelling, bleeding, fibrosis, other issues, or a combination thereof. Further, the fluid leaked into the retina may distort the vision of a subject immediately, and over time, can damage the retina itself, for example, by causing the loss of photoreceptors in the retina. The fluid may also cause the macula to separate from its base, resulting in severe and fast vision loss.

Treatment of nAMD typically involves an anti-vascular endothelial growth factor (anti-VEGF) therapy (e.g., an anti-VEGF drug such as ranibizumab). The retina's response to such treatment is at least partially subject specific, such that different subjects may respond differently to the same type of anti-VEGF drug. Faricimab is a newer treatment for nAMD and is a monoclonal antibody treatment. Both anti-VEGF therapies and monoclonal antibody treatments such as faricimab are typically administered via intravitreal injections, which can, in some instances, be expensive and be associated with side effects or complications (e.g., red eye, sore eye, infection, blindness, etc.). The number or frequency of the injections can also be burdensome on patients and lead to decreased control of the disease.

### SUMMARY

In one or more embodiments, a method is provided for predicting a selected treatment regimen for a subject with neovascular age-related macular degeneration (nAMD). Baseline data for a subject diagnosed with nAMD is received. A plurality of predictor inputs for an outcome predictor is formed using the baseline data and regimen data for a plurality of treatment regimens. The plurality of predictor inputs comprises a different predictor input for each of the plurality of treatment regimens, and wherein the outcome predictor comprises at least one machine learning model. A plurality of treatment scores for the plurality of treatment regimens is generated via the outcome predictor, using the plurality of predictor inputs. One of the plurality of treatment regimens is selected as a selected treatment regimen for the subject based on the plurality of treatment scores.

In one or more embodiments, a method is provided for predicting a selected treatment regimen for a subject with nAMD. Clinical data and imaging data may be received for a subject diagnosed with nAMD for a baseline point in time. Retinal feature data is generated using the imaging data. The retinal feature data comprises at least one of a pathology-related feature, a layer-related volume feature, or a layer-related thickness. A plurality of treatment scores for a plurality of treatment regimens is generated via an outcome predictor comprising at least one machine learning model. One of the plurality of treatment regimens is selected as a selected treatment regimen for the subject based on the plurality of treatment scores.

In one or more embodiments, a system is provided for predicting a selected treatment regimen for a subject diagnosed with neovascular age-related macular degeneration (nAMD). The system comprises a memory containing machine readable medium comprising machine executable code, and a processor coupled to the memory. The processor configured to execute the machine executable code to cause the processor to receive baseline data for a subject diagnosed with nAMD. The processor configured to execute the machine executable code to cause the processor to form a plurality of predictor inputs for an outcome predictor using the baseline data and regimen data for a plurality of treatment regimens. The plurality of predictor inputs comprises a different predictor input for each of the plurality of treatment regimens. The outcome predictor comprises at least one machine learning model. The processor configured to execute the machine executable code to cause the processor to generate, via the outcome predictor, a plurality of treatment scores for the plurality of treatment regimens using the plurality of predictor inputs. The processor configured to execute the machine executable code to cause the processor to select one of the plurality of treatment regimens as a selected treatment regimen for the subject based on the plurality of treatment scores.

In some embodiments, a system is provided that includes one or more data processors and a non-transitory machine-readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

### BRIEF DESCRIPTION OF THE DRAWINGS

The present disclosure is described in conjunction with the appended figures:

FIG. 1 is a block diagram of a prediction system 100 in accordance with various embodiments.

FIG. 2 is a block diagram of outcome prediction system 110 from FIG. 1 in accordance with one or more embodiments.

FIG. 3 is a schematic diagram of an exemplary workflow for selecting a treatment regimen to use for a subject with neovascular age-related macular degeneration (nAMD) in accordance with one or more embodiments.

FIG. 4 is a flowchart of a process for selecting a treatment regimen to use for a subject with neovascular age-related macular degeneration (nAMD) in accordance with one or more embodiments.

FIG. 5 is a flowchart of a process for selecting a treatment regimen to use for a subject with neovascular age-related macular degeneration (nAMD) in accordance with one or more embodiments.

FIG. 6 is a table showing the statistical results for the different models in accordance with one or more embodiments.

FIG. 7 is a block diagram of a computer system in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

Neovascular age-related macular degeneration (nAMD) may be treated with an anti-VEGF treatment, an antibody treatment, another type of treatment, or a combination thereof. Two examples of anti-VEGF treatments are ranibizumab and aflibercept, which may be administered via intravitreal injection. One example of an antibody treatment is a monoclonal antibody treatment, faricimab, that targets the vascular endothelial growth factor (VEGF) and angiopoietin-2 inhibitor. Typically, nAMD treatments are administered via injection (e.g., intravitreal injection) at a frequency ranging from about every four weeks to about eight weeks. Some patients, however, may not require such frequent injections.

The frequency of the treatments may be generally burdensome to patients and may contribute to decreased disease control in the real-world. For example, after an initial phase of treatment, patients may be scheduled for regular monthly visits over a pro re nata (PRN) or as needed period of time. This PRN period of time may be, for example, 21 to 24 months, or some other number of months. Traveling to a clinic for monthly visits during the PRN period of time may be burdensome for patients who do not need frequent treatments. For example, it may be overly burdensome to travel for monthly visits when the patient will only need five or fewer injections during the entire PRN period. Accordingly, patient compliance with visits may decrease over time, leading to reduced disease control.

Further, treatment response for neovascular age-related macular degeneration (nAMD) varies by subject or patient. For example, different subjects may react differently to different injection frequencies associated with the same type of treatment. As one example, two subjects given the same treatment according to the same injection frequency may experience different levels of vision improvement. In some examples, two subjects may need different numbers of injections over time in order to achieve the same or similar vision improvement. In other examples, two subjects may need two different types of treatment in order to achieve the same or similar vision improvement.

Thus, the embodiments described herein recognize that it may be desirable to have a way of maximizing vision improvement in a subject with the lowest number of or lowest frequency of injections. A treatment regimen may identify a treatment (e.g., anti-VEGF therapy, monoclonal antibody therapy, etc.) and at least one of an administration frequency, a dosage schedule, a monitoring schedule, or a combination thereof for the treatment. The administration frequency may be, for example, an injection frequency (e.g., for intravitreal injections). The dosage schedule may be, for example, the dosage amount per injection, which may remain constant or may change over time (e.g., a different dosage for earlier injections as compared to later injections). The monitoring schedule may be, for example, a schedule for one or more monitoring visits or imaging sessions to assess treatment progress.

The embodiments described herein provide methods and systems for selecting a treatment regimen for a given subject having neovascular age-related macular degeneration (nAMD) to maximize vision improvement in the subject with the lowest number of or frequency of injections. For example, multiple treatment regimens may be analyzed to determine the expected treatment response (or outcome) of each treatment regimen for a subject. The treatment regimen that provides the best response (or outcome) with the lowest treatment burden may be used as the selected treatment regimen. In some cases, the selected treatment regimen may be referred to as an optimal treatment regimen for the subject.

In one or more embodiments, various types of baseline data (e.g., baseline clinical data, baseline optical coherence tomography (OCT) image data, feature data extracted from baseline OCT image data, a combination thereof, etc.) are processed via one or more machine learning models to predict treatment response for each of a plurality of treatment regimens. This prediction may be in the form of, for example, a treatment score. The treatment score may be, for example, a visual acuity measurement for a future point in time (e.g., 3 months, 6 months, 7 months, 8 months, 9 months, 10 months, 11 months, 12 months, 13 months, 15 months, 18 months, 20 months, 24 months, or some other number of months, days, or years after treatment or a first dose of treatment). In other examples, the treatment score may be some other type of measurement of or metric indicative of vision health.

In some cases, there may be no difference between the treatment responses (or outcomes) of two or more treatment regimens or any difference may be minimal (e.g., below a selected threshold). In these cases, the treatment regimen that has the least treatment burden may be selected as the selected treatment regimen. Treatment burden may be determined based on, for example, without limitation, at least one of the number of and/or frequency of injections, a number of or frequency of follow-up visits that is recommended or required, the potential for one or more side effects or complications, dosage strength, or some other type of factor that makes treatment management or maintenance more difficult.

Recognizing and taking into account the importance and utility of a methodology and system that can provide the improvements described above, the embodiments described herein enable using machine learning models to predict a treatment regimen for a subject diagnosed with nAMD that will maximize vision improvement with the lowest number of or frequency of injections. Having the capability personalize the treatment regimen selected for a subject based on subject's baseline status will help support clinicians in making informed, personalized treatment decisions that can better benefit such subjects. Further, making such determinations based on treatment outcomes predicted by machine learning models may reduce the overall computing resources that might otherwise be needed to analyze treatment regimens and identify a selected treatment regimen.

### II. System for Selecting a Treatment Regimen for a Subject With nAMD

### II.A. Overview

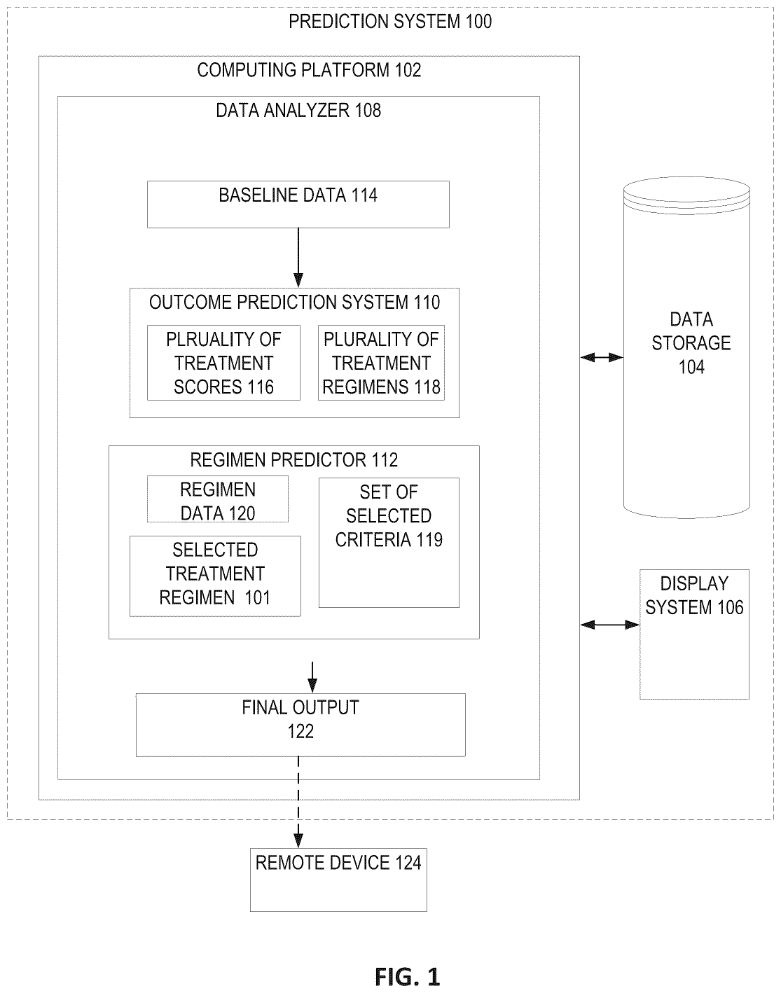

FIG. 1 is a block diagram of a prediction system 100 in accordance with various embodiments. Prediction system 100 may be used to predict selected treatment regimen 101 that will maximize vision improvement while minimizing treatment burden for one or more subjects who have been diagnosed with AMD and, in particular, nAMD. Treatment of such patients may include, for example, but is not limited to, an anti-VEGF treatment, an antibody treatment, another type of treatment, or a combination thereof. The anti-VEGF treatment may include, for example, ranibizumab, which may be administered via intravitreal injection. The antibody treatment may be, for example, but is not limited to, a monoclonal antibody treatment that targets the vascular endothelial growth factor (VEGF) and angiopoietin-2 inhibitor. In one or more embodiments, the antibody treatment includes faricimab.

As previously described, a treatment regimen may identify a treatment (e.g., anti-VEGF therapy, monoclonal antibody therapy, etc.) and at least one of an administration frequency, a dosage schedule, a monitoring schedule, or a combination thereof for the treatment. A treatment regimen may also be referred to as a treatment arm in some instances. The administration frequency may be, for example, an injection frequency (e.g., for intravitreal injections). The dosage schedule may be, for example, the dosage amount per injection, which may remain constant or may change over time (e.g., a different dosage for earlier injections as compared to later injections). The monitoring schedule may be, for example, a schedule for one or more monitoring visits or imaging sessions to assess treatment progress.

In one or more embodiments, selected treatment regimen 101 may be the treatment regimen that maximizes vision improvement in the subject while minimizing treatment burden. Treatment burden may be determined based on, for example, without limitation, at least one of the number of and/or frequency of injections, a number of or frequency of follow-up visits that is recommended or required, the potential for one or more side effects or complications, dosage strength, or some other type of factor that makes treatment management or maintenance more difficult. As one example, selected treatment regimen 101 may be the treatment regimen that provides the best treatment response (or outcome), while having the fewest number of injections or the lowest frequency of injections. In some cases, selected treatment regimen 101 may be referred to as an optimal treatment regimen.

Prediction system 100 includes computing platform 102, data storage 104, and display system 106. Computing platform 102 may take various forms. In one or more embodiments, computing platform 102 includes a single computer (or computer system) or multiple computers in communication with each other. In other examples, computing platform 102 takes the form of a cloud computing platform. In some examples, computing platform 102 takes the form of a mobile computing platform (e.g., a smartphone, a tablet, a smartwatch, etc.).

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together. Data storage 104 may include, for example, but is not limited to, a database, a spreadsheet, a server, cloud-based storage, or combination thereof.

Prediction system 100 includes data analyzer 108, which may be implemented using hardware, software, firmware, or a combination thereof. In one or more embodiments, data analyzer 108 is implemented in computing platform 102.

Data analyzer 108 includes outcome prediction system 110 and regimen predictor 112, each of which may be implemented using hardware, software, firmware, or a combination thereof. In some embodiments, outcome prediction system 110 and regimen predictor 112 may be implemented as separate modules. In other embodiments, at least a portion of regimen predictor 112 may be implemented as part of outcome prediction system 110.

Outcome prediction system 110 uses baseline data 114 to generate (or predict) plurality of treatment scores 116 for a corresponding plurality of treatment regimens 118. In some embodiments, baseline data 114 may be at least partially received from a source external to prediction system 100 over one or more communications links (e.g., wired communications links, wireless communications links, optical communications links, etc.). In one or more embodiments, baseline data 114 is at least partially retrieved from data storage 104.

Treatment regimens 118 may include two treatment regimens, three treatment regimens, four treatment regimens, or some other number of treatment regimens that may be implemented for the subject. Treatment regimens 118 may differ with respect to treatment type, dosage schedule, medication strength or concentration, the number of injections, injection frequency, a monitoring schedule, or a combination thereof. In this manner, two different treatment regimens may correspond to a same treatment but may differ by other factors (e.g., dosage and/or injection frequency).

Treatment scores 116 may include a corresponding treatment score for each of treatment regimens 118. Each of treatment scores 116 may be a score that indicates treatment response or outcome. As one example, a higher treatment score indicates a better response (e.g., a greater improvement to vision), while a lower treatment score indicates a poorer response. In other examples, a lower treatment score indicates a better response (e.g., a greater improvement to vision), while a higher treatment score indicates a poorer response.

The treatment score may indicate, for example, without limitation, at least one of a predicted visual acuity measurement (e.g., a predicted best corrected visual acuity (BCVA)), a predicted changed in visual acuity (e.g., a predicted change in BCVA), a predicted CST, a predicted reduction in CST, or some other type of response metric for a subject undergoing treatment. The treatment score may be generated for a selected point in time after treatment such as, for example, without limitation, 6 months, 9 months, 12 months, 24 months, or some other amount of time after treatment.

In one or more embodiments, the treatment scores 116 may be visual acuity measurements (e.g., BCVA) that are predicted for the subject at some future point in time after treatment or after a first dose of treatment. This future point in time may be, for example, 3 months, 6 months, 7 months, 8 months, 9 months, 10 months, 11 months, 12 months, 13 months, 15 months, 18 months, 20 months, 24 months, or some other number of months, days, or years. In one or more embodiments, treatment scores 116 may be metrics representative of or generated based on visual acuity measurements that are predicted for the subject at the future point in time.

Baseline data 114 may include data for a baseline point in time. The baseline point in time may be, for example, a point in time prior to treatment or a point in time concurrent with a first dose of a treatment (e.g., day one of treatment). In other embodiments, a point in time during treatment (e.g., 1 week, 2 weeks, 4 weeks, 1 month, 2 months, 3 months, etc. after an x^{th} dose of treatment). Examples of different types of baseline data are described in greater detail in Section II.B below.

Outcome prediction system 110 may be implemented in different ways to process baseline data 114 and generate treatment scores 116. Examples of implementations for outcome prediction system 110 are described in greater detail in Section II.B below.

Regimen predictor 112 may be used to identify selected treatment regimen 101 for use with the subject based on plurality of treatment scores 116. In one or more embodiments, regimen predictor 112 uses set of selected criteria 119 to identify one of treatment regimens 118 as selected treatment regimen 101. Set of selected criteria 119 may include various criteria. For example, set of selected criteria 119 may include a score criterion, a set of treatment burden criteria, or both. The set of treatment burden criteria may include, for example, without limitation, at least one of a fewest number of injections, a lowest frequency of injections, a lowest dosage, a lowest drug strength, a reduced amount of monitoring, or reduced side effects.

In one or more embodiments, a score criterion is a criterion for the treatment score. For example, the score criterion may be a highest treatment score or a lowest treatment score. In some embodiments, the score criterion may be for a subset of the treatment scores 116 (e.g., the subset of treatment scores 116 that fall within a selected range, the subset of treatment scores that are above a selected threshold, the subset of treatment scores that are below a selected threshold, or some other subset). In these examples, the score criterion may be a highest treatment score or a lowest treatment score of the subset.

In other embodiments, regimen predictor 112 may compute new scores based on treatment scores 116. These new scores may be a different type of indication of treatment response or outcome in some cases. In these examples, the score criterion may be for these computed scores. For example, the score criterion may be a highest computed score or a lowest computed score. In some cases, the score criterion may be for a subset of the computed scores (e.g., the subset of computed scores that fall within a selected range, the subset of computed scores that are above a selected threshold, the subset of computed scores that are below a selected threshold, or some other subset).

In one or more embodiments, regimen predictor 112 may identify the treatment regimen of treatment regimens 118 that most fully satisfies set of selected criteria 119 as selected treatment regimen 101 for use with the subject. As one example, regimen predictor 112 may first determine whether a single treatment regimen of the treatment regimens 118 or multiple ones of the treatment regimens meet the score criterion. If a single treatment regimen meets the score criterion, regimen predictor 112 identifies this single treatment regimen as selected treatment regimen 101, thereby predicting that selected treatment regimen 101 will maximize vision improvement while minimizing treatment burden for the subject. If, however, multiple treatment regimens meet the score criterion, regimen predictor 112 identifies the treatment regimen that meets the set of treatment burden criteria for the subject as selected treatment regimen 101.

For example, when the score criterion is a highest treatment score, regimen predictor 112 may select the treatment regimen having the highest treatment score as selected treatment regimen 101. However, in certain instances, two or more treatment regimens of treatment regimens 118 may tie. For example, two or more treatment regimens may tie by both sharing the highest treatment score. As another example, two or more treatment regimens may tie by having the two highest treatment scores with a difference between the two treatment scores that is below a selected threshold.

In the case of such a tie, regimen predictor 112 may select the treatment regimen that meets the set of treatment burden criteria (e.g., the one having the least treatment burden for the subject) as selected treatment regimen 101. In one or more embodiments, regimen predictor 112 uses regimen data 120 to identify which of the tied treatment regimens meets the set of treatment burden criteria (e.g., imposes the least burden on the subject).

Regimen data 120 may identify the particulars of each of the treatment regimens 118. Regimen data 120 may identify, for example, without limitation, the type of treatment and at least one of an administration frequency, a dosage schedule, a monitoring schedule, or a combination thereof for the treatment. In some cases, regimen data 120 may additionally identify other information about each of the treatment regimens 118. For example, regimen data 120 may identify side effect or complication information, dosage strength, and/or information related to the burden imposed by the given treatment regimen on a given subject.

Data analyzer 108 may use selected treatment regimen 101 and, in some cases, plurality of treatment scores 116, regimen data 120, or both, to form final output 122. Final output 122 may identify, for example, selected treatment regimen 101. In some cases, final output 122 may include the portion of information in regimen data 120 related to selected treatment regimen 101. In some examples, final output 122 includes the treatment scores 116 for all of the treatment regimens 118. These treatment scores 116 may be presented in chart form, in a graph, in a table, in a ranked list, or a combination thereof. In other examples, final output 122 includes only the treatment score corresponding to selected treatment regimen 101. In still other examples, final output 122 includes only a selected number of the highest (or alternatively, lowest) treatment scores. For example, final output 122 may include the top two treatment scores.

In one or more embodiments, final output 122 includes other information generated based on selected treatment regimen 101. For example, the prediction of selected treatment regimen 101 may be used to include or exclude subjects from a clinical trial. If, for example, a subject is being assigned to a clinical trial for one treatment regimen out of multiple possible treatment regimens, data analyzer 108 may be used to generate final output 122 that indicates which clinical trial would be expected to be most successful for the subject with the lowest treatment burden. In this manner, final output 122 may be used to improve overall treatment management as well as clinical trial population enrichment.

In one or more embodiments, at least a portion of final output 122 or a graphical representation of at least a portion of final output 122 is displayed on display system 106. In some embodiments, at least a portion of final output 122 or a graphical representation of at least a portion of final output 122 is sent to remote device 124 (e.g., a mobile device, a laptop, a server, a cloud, etc.).

### II.B. Exemplary Outcome Predictor

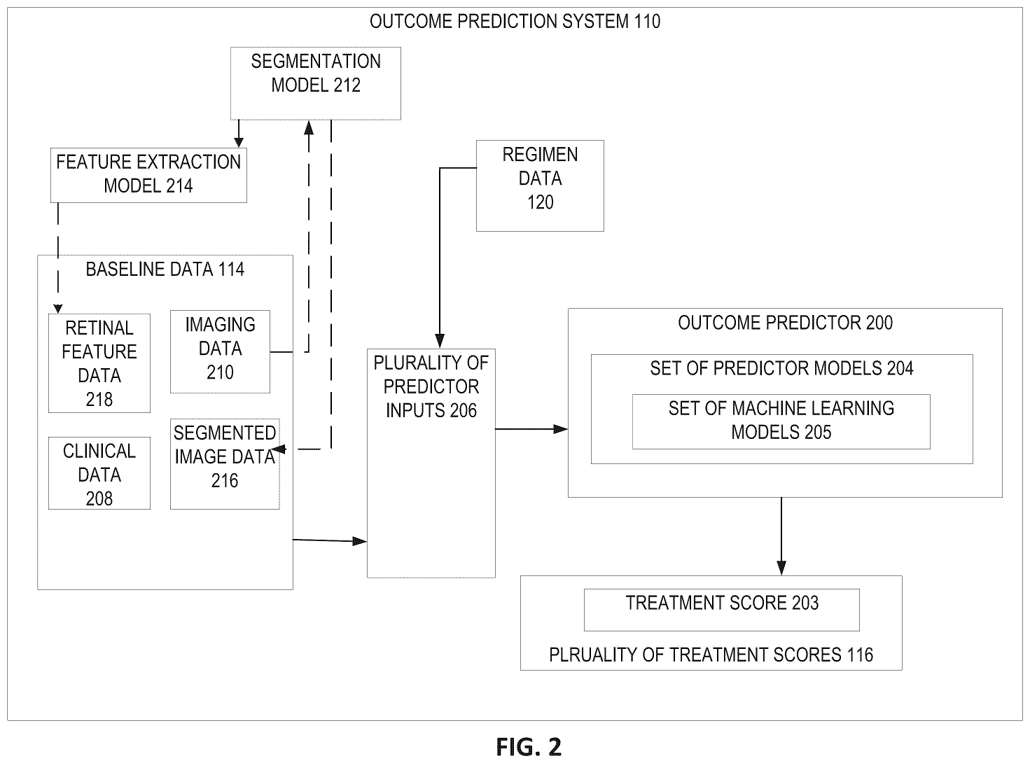

FIG. 2 is a block diagram of outcome prediction system 110 from FIG. 1 in accordance with one or more embodiments. Outcome prediction system 110 is described with continuing reference to the elements shown in FIG. 1. Outcome prediction system 110, as described above in Section II.A, is used to predict plurality of treatment scores 116 for a corresponding plurality of treatment regimens 118. Outcome prediction system 110 may include outcome predictor 200. Outcome predictor 200 may include, for example, set of predictor models 204. Set of predictor models 204 may be one or more predictor models. In one or more embodiments, each predictor model of set of predictor models 204 comprises one or more machine learning models. In this manner, set of predictor models 204 may be comprised of set of machine learning models 205. When set of predictor models 204 includes multiple predictor models, these predictor models may be implemented in a same or similar manner. In some cases, each of these predictor models may be implemented using a same machine learning model or a same machine learning architecture.

In one or more embodiments, outcome predictor 200 may be used to predict the treatment scores 116 for the treatment regimens 118 described in FIG. 1. Treatment score 203 may be one example of one of treatment scores 116. Treatment score 116 may be or may indicate at least one of a predicted visual acuity measurement (e.g., a predicted best corrected visual acuity (BCVA)), a predicted changed in visual acuity (e.g., a predicted change in BCVA), a predicted CST, a predicted reduction in CST, or some other type of response metric for a subject undergoing treatment. Treatment score 203 may be generated for a selected point in time after treatment such as, for example, without limitation, 6 months, 9 months, 12 months, 24 months, or some other amount of time after treatment.

Outcome prediction system 110 may receive and, in some cases, also generate baseline data 114. Baseline data 114 may be used to generate plurality of predictor inputs 206 that are processed by outcome predictor 200 to generate treatment scores 116. Plurality of predictor inputs 206 may be generated for treatment regimens 118 such that plurality of predictor inputs 206 includes one predictor input for each of the treatment regimens 118.

In one or more embodiments, baseline data 114 includes, but is not limited to, clinical data 208, imaging data 210, or a combination thereof for a baseline point in time. Clinical data 208 may include, for example, without limitation, at least one of baseline demographic data, a baseline visual acuity measurement, a baseline central subfield thickness (CST) measurement, a baseline low-luminance deficit (LLD), or some other type of baseline measurement. The baseline demographic data may include, for example, without limitation, at least one of age, sex, or another type of demographic metric. The baseline visual acuity measurement may be, for example, a best corrected visual acuity (BCVA) measurement. The baseline CST measurement may be, for example, in micrometers. The baseline LLD may be the difference between a baseline BCVA measurement and a baseline low-luminance visual acuity (LLVA) measurement.

Imaging data 210 may include one or more types of imaging data for the baseline point in time. For example, imaging data 210 may include optical coherence tomography (OCT) image data, color fundus image data, infrared image data, near-infrared image data, fundus autofluorescence image data, fluorescein angiography image data, another type of three-dimensional imaging data, or a combination thereof. In one or more embodiments, imaging data 210 includes three-dimensional OCT imaging data, data that is extracted from OCT images (e.g., OCT en-face images), tabular data extracted from OCT images, some other form of imaging data, or a combination thereof. The OCT imaging data may include, for example, spectral domain OCT (SD-OCT) B-scans that together form an OCT volume scan for the subject.

In some embodiments, outcome prediction system 110 may include other models that can be used to generate baseline data 114 for use in forming plurality of predictor inputs 206. For example, outcome prediction system 110 may include segmentation model 212, feature extraction module 214, or both.

Segmentation model 212 may be used to perform automated segmentation of imaging data 210 (e.g., of OCT imaging data) to form segmented image data 216. Segmentation model 212 may be implemented using a deep learning model (e.g., one or more neural networks). For example, segmentation model 212 may be implemented using one or more convolutional neural networks. In one or more embodiments, segmentation model 212 includes a U-Net. In some embodiments, at least a portion of segmented image data 216 is used to form plurality of predictor inputs 206 for outcome predictor 200. In some embodiments, at least a portion of segmented image data 216 may be sent to feature extraction module 214 for further processing. For example, in some cases, each of the B-scans (e.g., 128 B scans) that form an OCT volume scan in imaging data 210 may be segmented to form segmented image data 216 (e.g., 128 segmented images). But, in certain cases, only a portion (e.g., 20, 30, 40, 50, etc.) of the segmented images are sent to feature extraction module 214 for processing or used to form plurality of predictor inputs 206.

Segmentation via segmentation model 212 includes the detection and identification of one or more retinal (e.g., retina-associated) elements in a retinal image. A segmented image in segmented image data 216 identifies one or more retinal (e.g., retina-associated) elements on the segmented image using one or more graphical indicators. The segmented image may be a representation of an OCT image that identifies the one or more retinal elements or may be an OCT image on which the one or more retinal elements have been identified.

For example, one or more color indicators, shape indicators, pattern indicators, shading indicators, lines, curves, markers, labels, tags, text features, other types of graphical indicators, or a combination thereof may be used to identify the portion(s) (e.g., by pixel) of the image that have been identified as a retinal element. As one specific example, a group of pixels may be identified as capturing a particular retinal fluid (e.g., IRF or SRF). A segmented image may identify this group of pixels using a color indicator. For example, each pixel of the group of pixels may be assigned a color that is unique to the particular retinal fluid and thereby assigns each pixel to the particular retinal fluid. As another example, the segmented image may identify the group of pixels by applying a patterned region or shape (continuous or discontinuous) over the group of pixels.

A retinal element may be comprised of at least one of a retinal layer element or a retinal pathological element. Detection and identification of one or more retinal layer elements may be referred to as layer element (or retinal layer element) segmentation. Detection and identification of one or more retinal pathological elements may be referred to as pathological element (or retinal pathological element) segmentation.

A retinal layer element may be, for example, a retinal layer or a boundary associated with a retinal layer. Examples of retinal layers include, but are not limited to, an internal limiting membrane (ILM) layer, a retinal nerve fiber layer, a ganglion cell layer, an inner plexiform layer, an inner nuclear layer, an outer plexiform layer, an outer nuclear layer, an external limiting membrane (ELM) layer, a photoreceptor layer(s), a retinal pigment epithelial (RPE) layer, a layer of RPE detachment, a Bruch's membrane (BM) layer, a choriocapillaris layer, a choroidal stroma layer, an ellipsoid zone (EZ), and other types of retinal layer. In some cases, a retinal layer may be comprised of one or more layers. As one example, a retinal layer may be the interface between an outer plexiform layer and Henle's fiber layer (OPL-HFL). A boundary associated with a retinal layer may be, for example, an inner boundary of the retinal layer, an outer boundary of the retinal layer, a boundary associated with a pathological feature of the retinal layer (e.g., an inner or outer boundary of detachment of the retinal layer), or some other type of boundary. For example, a boundary may be an inner boundary of an RPE (IB-RPE) detachment layer, an outer boundary of the RPE (OB-RPE) detachment layer, or another type of boundary.

A retinal pathological element may include, for example, fluid (e.g., a fluid pocket), cells, solid material, or a combination thereof that evidences a retinal pathology (e.g., disease or condition such as AMD or diabetic macular edema). For example, the presence of certain retinal fluids may be a sign of nAMD. Examples of retinal pathological elements include, but are not limited to, intraretinal fluid (IRF), subretinal fluid (SRF), fluid associated with pigment epithelial detachment (PED), hyperreflective material (HRM), subretinal hyperreflective material (SHRM), intraretinal hyperreflective material (IHRM), hyperreflective foci (HRF), a retinal fluid pocket, drusen, a development of fibrosis, and a disruption. In some cases, a retinal pathological element may be a disruption (e.g., discontinuity, delamination, loss, etc.) of a retinal layer or retinal zone. For example, the disruption may be of the ellipsoid zone, of the ELM, of the RPE, or of another layer or zone. The disruption may represent damage to or loss of cells (e.g., photoreceptors) in the area of the disruption. In some examples, a retinal pathological element may be clear IRF, turbid IRF, clear SRF, turbid SRF, some other type of clear retinal fluid, some other type of turbid retinal fluid, or a combination thereof.

Feature extraction module 214 may be used to extract retinal feature data 218 from segmented image data 216 generated by segmentation model 212 or aggregate retinal feature data 218 from data received directly from segmentation model 212. In one or more embodiments, at least a portion of retinal feature data 218 may be used to form plurality of predictor inputs 206. Retinal feature data 218 may include, for example, without limitation, values for any number of or combination of features (e.g., quantitative retinal features). These features may include pathology-related features, layer-related volume features, layer-related thickness features, or a combination thereof. Examples of features include, but are not limited to, a maximum retinal layer thickness, a minimum retinal layer thickness, an average retinal layer thickness, a maximum height of a boundary associated with a retinal layer, a volume of a retinal fluid pocket, a length of a fluid pocket, a width of a fluid pocket, a number of retinal fluid pockets, and a number of hyperreflective foci. Thus, at least some of the features may be volumetric features. For example, the feature data may be derived for each selected OCT image (e.g., single OCT B scan) and then combined to form volume-wide values. In one or more embodiments, between 1 to 200 features may be included in retinal feature data 218.

In this manner, at least a portion of clinical data 208, at least a portion of imaging data 210, at least a portion of segmented image data 216, at least a portion of retinal feature data 218, or a combination thereof may be used to form each predictor input in plurality of predictor inputs 206 that is sent into outcome predictor 200 for processing. This information may be the same for each of plurality of predictor inputs 206.

Further, each of plurality of predictor inputs 206 may further include at least a portion of regimen data 120 for the corresponding treatment regimen. For example, when set of predictor models 204 includes a single predictor model (e.g., which may include one or more machine learning models), each predictor input of plurality of predictor inputs 206 may be fed into the single predictor model independently. Each predictor input includes the portion of regimen data 120 for its corresponding treatment regimen. In this manner, each of plurality of predictor inputs 206 is processed through the single predictor model independently (e.g., via a different forward pass) to generate treatment scores 116.

In other examples, when set of predictor models 204 includes a plurality of predictor models with each corresponding to a different one of treatment regimens 118, each of plurality of predictor models 206 may be sent into its corresponding predictor model to generate treatment scores 116 overall. In this manner, each of the plurality of predictor models is specifically tailored to generate a treatment score for a corresponding treatment regimen.

Outcome predictor 202 may be implemented in various different ways. In one or more embodiments, outcome predictor 202 may be implemented using any number of or combination of artificial intelligence models or machine learning models. Outcome predictor 202 may be comprised of multiple models and/or algorithms. For example, each predictor model of set of predictor models 204 may include any number or combination of regression models, linear models, random forest models, XGBoost algorithms, deep learning models (e.g., neural networks), and/or other types of machine learning models.

In one or more embodiments, outcome predictor 202 (e.g., one of set of predictor models 204) includes a deep learning model and a symbolic model (e.g., a classic machine learning model). In one or more embodiments, the deep learning model includes one or more neural networks, with at least one of these one or more neural networks being a deep learning neural network (or deep neural network (DNN)). In one or more embodiments, the symbolic model includes one or more models that use symbolic machine learning. The symbolic model may include, for example, without limitation, at least one of a linear model, a random forest model, an Extreme Gradient Boosting (XGBoost) algorithm, a Support Vector Machine (SVM) model, or another type of model or algorithm that uses symbolic learning.

In various embodiments, outcome predictor 202 may use a model stacking approach to generate treatment score 203. In a model stacking approach, for example, a first machine learning model (e.g., deep learning model) may be used to generate an intermediate treatment score that is sent as input into a second machine learning model (e.g., a symbolic model). The intermediate treatment score may be sent as input, along with other data (e.g., baseline clinical data) into the second machine learning model for processing. The second machine learning model then generates treatment score 203.

In various embodiments, outcome predictor 202 may use a model averaging approach to generate treatment score 203. In a model averaging approach, at least two different machine learning models may use plurality of predictor inputs 206 to generate intermediate treatment scores. These intermediate treatment scores may then be averaged (e.g., via equal weighting) to generate treatment score 203.

Together, outcome predictor 200 generates treatment scores 116. Treatment scores 116 may then be sent to regimen predictor 112, described above with respect to FIG. 1, to predict selected treatment regimen 101 for use with the subject.

### II.C. Exemplary Workflow for Predicting Selected Treatment Regimen

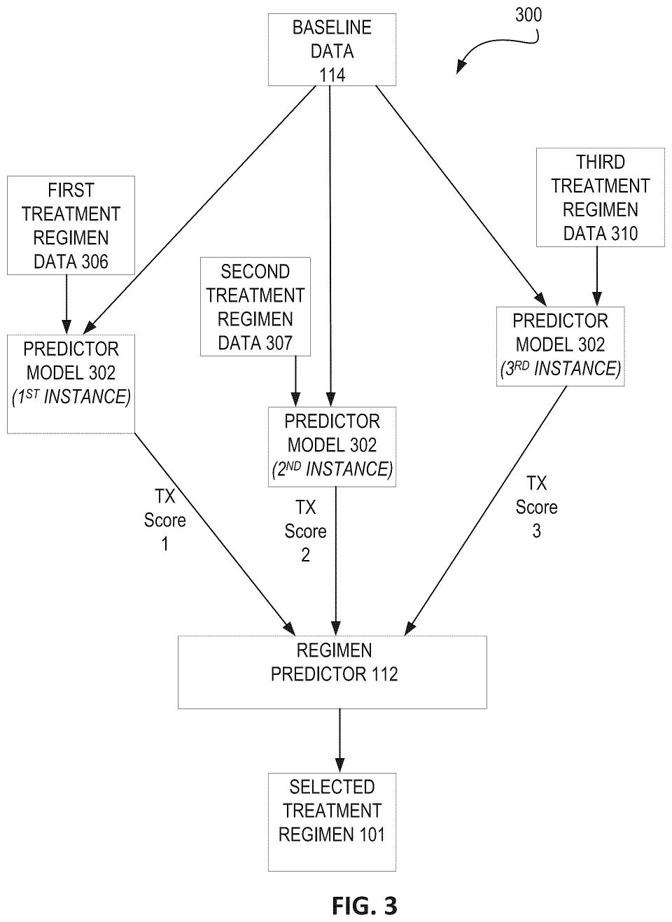

FIG. 3 is a schematic diagram of an exemplary workflow for the prediction of a selected treatment regimen. Workflow 300 is described with continuing reference to elements in FIGS. 1 and 2. Workflow 300 shows how baseline data 114 may be processed using machine learning to predict selected treatment regimen 101. Predictor model 302 may be one example of a predictor model in set of predictor models 204 in outcome predictor 200 in FIG. 2. Predictor model 302 may be used in a plurality of instances (e.g., multiple cycles of processing) for a plurality of treatment regimens. For example, for three different treatment regimens, predictor model 302 may be run three different times using three different inputs. Predictor model 302 may be implemented using a machine learning model (e.g., a linear regression model, an SVM, an XGBoost algorithm, random forest, etc.).

Baseline data 114 may be used as input for each instance of predictor model 302. In FIG. 3, baseline data 114 includes a baseline visual acuity measurement, a baseline CST measurement, baseline age, and sex. First treatment regimen data 306 may be used as input for the first instance of predictor model 302. Second treatment regimen data 307 may be used as input for the second instance of predictor model 302. Third treatment regimen data 310 may be used as input for the third instance of predictor model 302. First treatment regimen data 306, second treatment regimen data 307, and third treatment regimen data 310 may be different portions of regimen data 120 described with respect to FIGS. 1 and 2, each identifying a different corresponding treatment regimen (e.g., a different one of treatment regimens 118 in FIG. 1).

During each instance or run cycle (or forward pass), predictor model 302 processes the input that has been received. Predictor model 302 generates a treatment score for the corresponding designated treatment regimen for that instance. For example, in the first instance, predictor model 302 generates treatment score 312; in the second instance, predictor model 302 generates treatment score 314; and in the third instance, predictor model 302 generates treatment score 316.

These treatment scores are processed by regimen predictor 112. Regimen predictor 112 uses a set of selected criteria that may include a score criterion to identify selected treatment regimen 101. For example, regimen predictor 112 may use a maximum value function that identifies the maximum of the inputs provided to the maximum value function. In other words, regimen predictor 112 identifies the treatment score that meets the score criterion of being the maximum treatment score.

### III. Exemplary Methodologies for Predicting a Selected Treatment Regimen

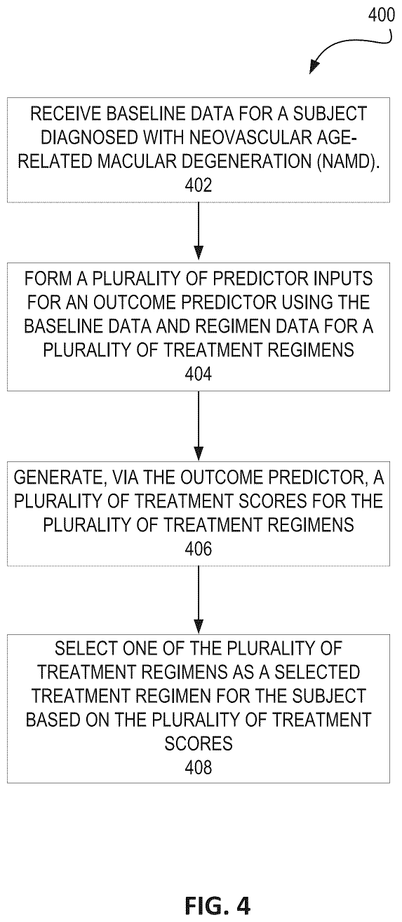

FIG. 4 is a flowchart of a process for predicting a selected treatment regimen in accordance with one or more embodiments. In one or more embodiments, process 400 may be implemented using prediction system 100 described in FIG. 1 and/or outcome prediction system 110 described in FIGS. 1-2. Process 400 includes various steps and may be described with continuing reference to FIGS. 1-2. One or more steps that are not expressly illustrated in FIG. 4 may be included before, after, in between, or as part of the steps of process 400. In some embodiments, process 400 may begin with step 402.

Step 402 includes receiving baseline data for a subject diagnosed with neovascular age-related macular degeneration (nAMD). The baseline data may be, for example, baseline data 114 in FIGS. 1-2. For example, the baseline data may include clinical data, imaging data, segmented image data, retinal feature data, or a combination thereof for a baseline point in time. The clinical data may include, for example, at least tone of a baseline age, sex, a baseline CST, a baseline visual acuity measurement (e.g., baseline BCVA), or some other type of baseline factor.

Step 404 includes forming a plurality of predictor inputs for an outcome predictor using the baseline data and regimen data for a plurality of treatment regimens. The plurality of predictor inputs comprises a different predictor input for each of the plurality of treatment regimens. The outcome predictor comprises at least one machine learning model. For example, the outcome predictor may include one or more predictor models, each of which may be comprised of least one of a linear regression model, a random forest (RF) model, a Gradient Boosting Machine (GBM) model, an Extreme Gradient Boosting (XGBoost), or a Support Vector Machine (SVM) model. In other embodiments, an outcome predictor may include a deep learning model (e.g., one or more neural networks). The outcome predictor may be, for example, outcome predictor 200 in FIG. 2.

The plurality of predictor inputs may be, for example, without limitation, plurality of predictor inputs 206 in FIG. 2. The regimen data may be, for example, regimen data 120 in FIGS. 1-2. The regimen data for a corresponding one of the plurality of treatment regimens identifies a treatment and at least one of an administration frequency, a dosage schedule, or a monitoring schedule for the treatment. Further, each of the plurality of predictor inputs 206 corresponds to a different one of the plurality of treatment regimens by including treatment regimen data that identifies that particular treatment regimen.

The predictor input for a corresponding treatment regimen may also include, for example, without limitation, clinical data and retinal feature data. The retinal feature data may be included in baseline data is received in step 402. In other examples, the retinal feature data may be extracted from segmented image data that is included in the baseline data. In still other examples, the retinal feature data may be extracted from segmented image data that is generated based on the imaging data (e.g., baseline OCT volume scans) that is included in the baseline data received in step 402.

Step 406 includes generating, via the outcome predictor, a plurality of treatment scores for the plurality of treatment regimens. Each of the treatment scores may be, for example, a predicted visual acuity measurement (e.g., a predicted BCVA), a predicted changed in visual acuity (e.g., a predicted change in BCVA), a predicted CST, a predicted reduction in CST, or some other type of response metric for a subject undergoing treatment. The treatment score may be generated for a selected point in time after treatment such as, for example, without limitation, 6 months, 9 months, 12 months, 24 months, or some other amount of time after treatment. In one example, the treatment scores are predicted BCVA at month 9.

Step 408 includes selecting one of the plurality of treatment regimens as a selected treatment regimen for the subject based on the plurality of treatment scores. The selected treatment regimen maximizes vision improvement while minimizing treatment burden (e.g., number of injections, injection frequency). In some embodiments, step 408 includes determining whether a single treatment regimen of the plurality of treatment regimens or multiple treatment regimens of the plurality of treatment regimens meets a score criterion. The score criterion may be, for example, a highest (maximum) treatment score, a lowest (minimum) treatment score, a highest computed score (e.g., a score computed or otherwise derived from the treatment score), a lowest computed score, or some other type of treatment score. In some cases, a treatment score or a score computed based on the treatment score may include letters or other text. The score criterion may be selected to identify the best predicted treatment outcome or response based on the treatment score or computed score.

Multiple treatment regimen may meet a score criterion by tying. For example, two treatment scores may be tied by sharing the same value. In other cases, the scores may be tied by having a difference that is less than a selected threshold. For example, without limitation, a numerical difference of 1 or less may be considered a tie.

If a single treatment regimen meets the score criterion, step 408 may include selecting the single treatment regimen as the selected treatment regimen for the subject. If, however, multiple treatment regimens meet the score criterion, step 408 may include identifying a treatment regimen of the multiple treatment regimens that meets a set of treatment burden criteria for the subject as the selected treatment regimen. The set of treatment burden criteria may include, for example, without limitation, at least one of a fewest number of injections, a lowest frequency of injections, a lowest dosage, a lowest drug strength, a reduced amount of monitoring, or reduced side effects.

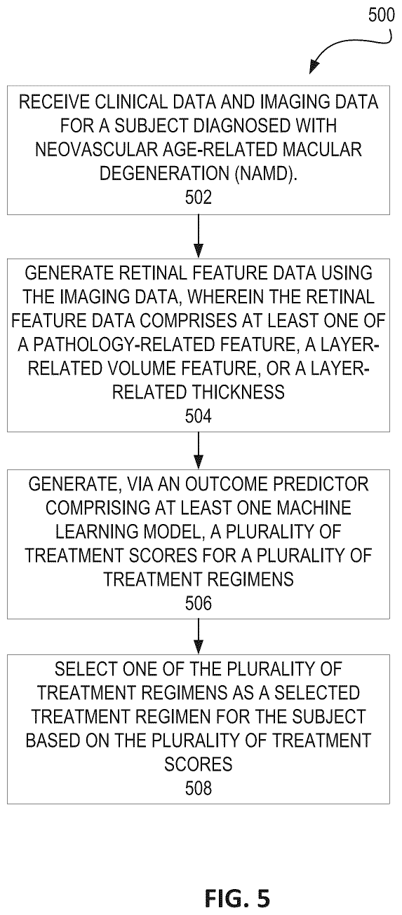

FIG. 5 is a flowchart of a process for predicting a selected treatment regimen in accordance with one or more embodiments. In one or more embodiments, process 500 may be implemented using prediction system 100 described in FIG. 1 and/or outcome prediction system 110 described in FIGS. 1-2. Process 500 includes various steps and may be described with continuing reference to FIGS. 1-2. One or more steps that are not expressly illustrated in FIG. 5 may be included before, after, in between, or as part of the steps of process 500. In some embodiments, process 500 may begin with step 502.

Step 502 includes receiving clinical data and imaging data for a subject diagnosed with neovascular age-related macular degeneration (nAMD) for a baseline point in time. The clinical data and imaging data may be, for example, clinical data 208 and imaging data 210 in FIG. 2.

Step 504 includes generating retinal feature data using the imaging data, wherein the retinal feature data comprises at least one of a pathology-related feature, a layer-related volume feature, or a layer-related thickness. The retinal feature data may be, for example, retinal feature data 218 in FIG. 2.

Step 506 includes generating, via an outcome predictor comprising at least one machine learning model, a plurality of treatment scores for a plurality of treatment regimens. In one or more embodiments, each of the machine learning models may be implemented using a regression model, XGBoost, an SVM model, random forest, and/or another type of machine learning model. Each of the treatment scores may be, for example, a predicted visual acuity measurement (e.g., a predicted BCVA), a predicted changed in visual acuity (e.g., a predicted change in BCVA), a predicted CST, a predicted reduction in CST, or some other type of response metric for a subject undergoing treatment. The treatment score may be generated for a selected point in time after treatment such as, for example, without limitation, 6 months, 9 months, 12 months, 24 months, or some other amount of time after treatment.

Step 508 includes selecting one of the plurality of treatment regimens as a selected treatment regimen for the subject based on the plurality of treatment scores. Step 508 may be implemented in a manner similar to step 408 of process 400 described above with respect to FIG. 4.

### IV. Exemplary Study Results

In one study, a dataset consisting of eyes for 324 subjects treated with ranibizumab at 0.5 mg Q4W (i.e., every 4 weeks). These subjects were treatment naïve prior to treatment with ranibizumab. Baseline BCVA for the subjects ranged from 20/40-20/320 (i.e., 73-24 according to Early Treatment Diabetic Retinopathy Severity (ETDRS) letters). All subjects were at or over the age of 50 years old.

Training was performed to predict the selected treatment regimen (e.g., optimal treatment regimen) for use with subjects based on benchmark features (e.g., age, sex, baseline BCVA, baseline CST). Training was also performed to identify the selected treatment regimen based on the benchmark features and retinal feature data derived from imaging data (e.g., from segmented images generated from SD-OCT images).

Each of a linear model, a random forest model, an XGBoost model, and a Support Vector Machine model were developed for predicting treatment score (e.g., a predicted BCVA at month 9) for both the benchmark features and the benchmark features plus retinal features. The regimen having the highest treatment score was selected as the selected treatment regimen. Model performance was evaluated using R² scores (i.e., coefficient of determination) in nested cross-validation (5-fold, repeated 10 times).

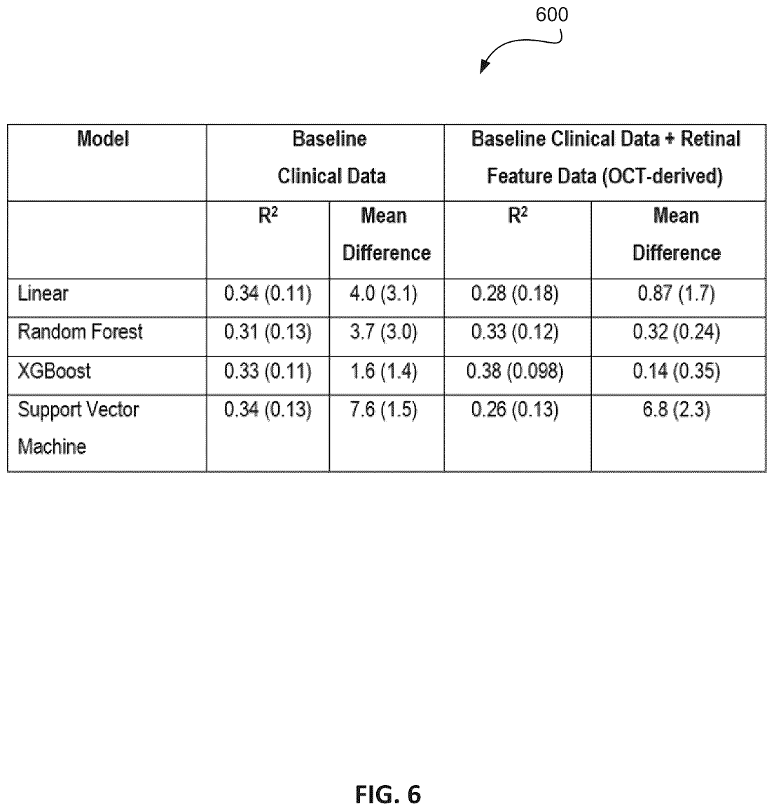

FIG. 6 is a table 600 showing the statistical results for the different models in accordance with one or more embodiments. Table 600 shows that using the linear model, the random forest model, the XGBoost model, and the Support Vector Machine model to compute treatment scores based on both the benchmark features and the image-derived retinal features performed well in this particular study, with XGBoost generally performing the best.

### V. Computer-Implemented System

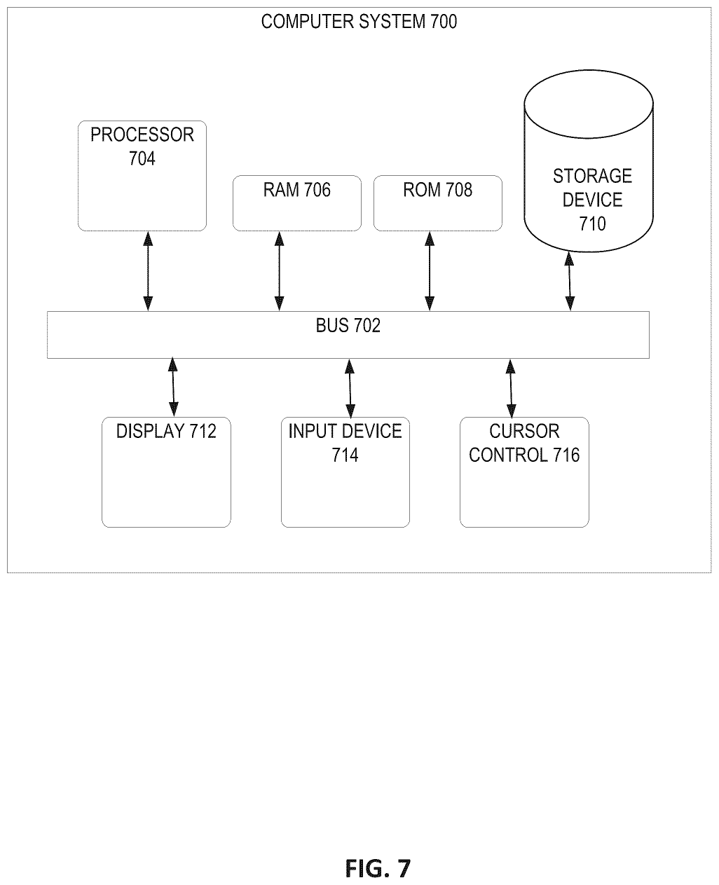

FIG. 7 is a block diagram illustrating an example of a computer system in accordance with various embodiments. Computer system 700 may be an example of one implementation for computing platform 102 described above in FIG. 1. In one or more examples, computer system 700 can include a bus 702 or other communication mechanism for communicating information, and a processor 704 coupled with bus 702 for processing information. In various embodiments, computer system 700 can also include a memory, which can be a random-access memory (RAM) 706 or other dynamic storage device, coupled to bus 702 for determining instructions to be executed by processor 704. Memory also can be used for storing temporary variables or other intermediate information during execution of instructions to be executed by processor 704. In various embodiments, computer system 700 can further include a read-only memory (ROM) 708 or other static storage device coupled to bus 702 for storing static information and instructions for processor 704. A storage device 710, such as a magnetic disk or optical disk, can be provided and coupled to bus 702 for storing information and instructions.

In various embodiments, computer system 700 can be coupled via bus 702 to a display 712, such as a cathode ray tube (CRT) or liquid crystal display (LCD), for displaying information to a computer user. An input device 714, including alphanumeric and other keys, can be coupled to bus 702 for communicating information and command selections to processor 704. Another type of user input device is a cursor control 716, such as a mouse, a joystick, a trackball, a gesture-input device, a gaze-based input device, or cursor direction keys for communicating direction information and command selections to processor 704 and for controlling cursor movement on display 712. This input device 716 typically has two degrees of freedom in two axes, a first axis (e.g., x) and a second axis (e.g., y), that allows the device to specify positions in a plane. However, it should be understood that input devices 716 allowing for three-dimensional (e.g., x, y and z) cursor movement are also contemplated herein.

Consistent with certain implementations of the present teachings, results can be provided by computer system 700 in response to processor 704 executing one or more sequences of one or more instructions contained in RAM 706. Such instructions can be read into RAM 706 from another computer-readable medium or computer-readable storage medium, such as storage device 710. Execution of the sequences of instructions contained in RAM 706 can cause processor 704 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 704 for execution. Such a medium can take many forms, including but not limited to, non-volatile media, volatile media, and transmission media. Examples of non-volatile media can include, but are not limited to, optical, solid state, magnetic disks, such as storage device 710. Examples of volatile media can include, but are not limited to, dynamic memory, such as RAM 706. Examples of transmission media can include, but are not limited to, coaxial cables, copper wire, and fiber optics, including the wires that comprise bus 702.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 704 of computer system 700 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 700 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 700, whereby processor 704 would execute the analyses and determinations provided by these engines, subject to instructions provided by any one of, or a combination of, the memory components RAM 706, ROM 708, or storage device 710 and user input provided via input device 714.

### VI. Exemplary Definitions and Context

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. In various cases, “subject” and “patient” may be used interchangeably herein.

Unless otherwise defined, scientific and technical terms used in connection with the present teachings described herein shall have the meanings that are commonly understood by those of ordinary skill in the art. Further, unless otherwise indicated by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and, in some cases, only one of the items in the list may be used. The item may be a particular object, thing, step, operation, process, or category. In other words, “at least one of” means any combination of items or number of items may be used from the list, but not all of the items in the list may be used. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming.

As used herein, an “artificial neural network” or “neural network” (NN) may refer to mathematical algorithms or computational models that mimic an interconnected group of artificial neurons that processes information based on a connectionistic approach to computation. Neural networks, which may also be referred to as neural nets, can employ one or more layers of linear units, nonlinear units, or both to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways; when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

### VII. RECITATION OF EXEMPLARY EMBODIMENTS

Embodiment 1: A method comprising: receiving baseline data for a subject diagnosed with neovascular age-related macular degeneration (nAMD); forming a plurality of predictor inputs for an outcome predictor using the baseline data and regimen data for a plurality of treatment regimens, wherein the plurality of predictor inputs comprises a different predictor input for each of the plurality of treatment regimens, wherein the outcome predictor comprises at least one machine learning model; generating, via the outcome predictor, a plurality of treatment scores for the plurality of treatment regimens using the plurality of predictor inputs; and selecting one of the plurality of treatment regimens as a selected treatment regimen for the subject based on the plurality of treatment scores.

Embodiment 2: The method of embodiment 1, wherein the selected treatment regimen maximizes vision improvement and minimizes an injection frequency.

Embodiment 3: The method of embodiment 1 or embodiment 2, wherein the selecting comprises: determining whether a single treatment regimen of the plurality of treatment regimens or multiple treatment regimens of the plurality of treatment regimens meets a score criterion.

Embodiment 4: The method of embodiment 3, wherein the selecting further comprises: responsive to the single treatment regimen meeting the score criterion, selecting the single treatment regimen as the selected treatment regimen for the subject; or responsive to the multiple treatment regimens meeting the score criterion, identifying a treatment regimen of the multiple treatment regimens that meets a set of treatment burden criteria for the subject as the selected treatment regimen.

Embodiment 5: The method of embodiment 4, wherein the set of treatment burden criteria comprises at least one of a fewest number of injections, a lowest frequency of injections, a lowest dosage, a lowest drug strength, a reduced amount of monitoring, or reduced side effects.

Embodiment 6: The method of any one of embodiments 1-5, wherein the generating comprises: processing, via the outcome predictor, each of the plurality of predictor inputs independently to generate the plurality of treatment scores.

Embodiment 7: The method of any one of embodiments 1-5, wherein: the outcome predictor comprises a plurality of predictor models; each of the plurality of predictor models comprises at least one machine learning model; and each of the plurality of predictor models is configured to generate a treatment score of the plurality of treatment scores for a corresponding treatment regimen of the plurality of treatment regimens using a predictor input of the plurality of predictor inputs that corresponds to the treatment regimen.

Embodiment 8: The method of any one of embodiments 1-7, wherein the baseline data comprises at least one of: optical coherence tomography (OCT) image data; clinical data that includes at least one of a visual acuity measurement, a central subfield thickness, a low-luminance deficit, age, or sex; or retinal feature data extracted from segmented image data that has been generated from OCT image data corresponding to a baseline point in time.

Embodiment 9: The method of any one of embodiments 1-8, wherein the regimen data for a corresponding one of the plurality of treatment regimens identifies a treatment and at least one of an administration frequency, a dosage schedule, or a monitoring schedule for the treatment.

Embodiment 10: The method of any one of embodiments 1-9, wherein each of the treatment scores is a predicted visual acuity measurement.

Embodiment 11: The method of any one of embodiments 1-10, wherein the at least one machine learning model comprises at least one of a linear regression model, a random forest (RF) model, a Gradient Boosting Machine (GBM) model, an Extreme Gradient Boosting (XGBoost), or a Support Vector Machine (SVM) model.

Embodiment 12: A method comprising: receiving clinical data and imaging data for a subject diagnosed with neovascular age-related macular degeneration (nAMD) for a baseline point in time; generating retinal feature data using the imaging data, wherein the retinal feature data comprises at least one of a pathology-related feature, a layer-related volume feature, or a layer-related thickness; generating, via an outcome predictor comprising at least one machine learning model, a plurality of treatment scores for a plurality of treatment regimens; selecting one of the plurality of treatment regimens as a selected treatment regimen for the subject based on the plurality of treatment scores.

Embodiment 13: The method of embodiment 12, wherein the selecting comprises: determining whether a single treatment regimen of the plurality of treatment regimens or multiple treatment regimens of the plurality of treatment regimens meets a score criterion.

Embodiment 14: The method of embodiment 13, wherein the selecting further comprises: responsive to the single treatment regimen meeting the score criterion, selecting the single treatment regimen as the selected treatment regimen for the subject; or responsive to the multiple treatment regimens meeting the score criterion, identifying a treatment regimen of the multiple treatment regimens that meets a set of treatment burden criteria for the subject as the selected treatment regimen.

Embodiment 15: The method of any one of embodiments 12-14, wherein the clinical data comprises at least one of a visual acuity measurement, a central subfield thickness, a low-luminance deficit, age, or sex for the baseline point in time and wherein the imaging data comprises optical coherence tomography (OCT) image data.

Embodiment 16: The method of any one of embodiments 12-15, wherein each of the treatment scores is a predicted visual acuity measurement.

Embodiment 17: The method of any one of embodiments 12-16, wherein the at least one machine learning model comprises at least one of a linear regression model, a random forest (RF) model, a Gradient Boosting Machine (GBM) model, an Extreme Gradient Boosting (XGBoost), or a Support Vector Machine (SVM) model.

Embodiment 18: A system for predicting a selected treatment regimen for a subject diagnosed with neovascular age-related macular degeneration, the system comprising: a memory containing machine readable medium comprising machine executable code; and a processor coupled to the memory, the processor configured to execute the machine executable code to cause the processor to: receive baseline data for a subject diagnosed with neovascular age-related macular degeneration (nAMD); form a plurality of predictor inputs for an outcome predictor using the baseline data and regimen data for a plurality of treatment regimens, wherein the plurality of predictor inputs comprises a different predictor input for each of the plurality of treatment regimens, wherein the outcome predictor comprises at least one machine learning model; generate, via the outcome predictor, a plurality of treatment scores for the plurality of treatment regimens using the plurality of predictor inputs; and select one of the plurality of treatment regimens as a selected treatment regimen for the subject based on the plurality of treatment scores.

Embodiment 19: The system of embodiment 18, wherein the selected treatment regimen maximizes vision improvement and minimizes an injection frequency.

Embodiment 20: The system of embodiment 18 or embodiment 19, wherein the processor is further configured to execute the machine executable code to cause the processor to: determine whether a single treatment regimen of the plurality of treatment regimens or multiple treatment regimens of the plurality of treatment regimens meets a score criterion; and responsive to the single treatment regimen meeting the score criterion, select the single treatment regimen as the selected treatment regimen for the subject; or responsive to the multiple treatment regimens meeting the score criterion, identify a treatment regimen of the multiple treatment regimens that meets a set of treatment burden criteria for the subject as the selected treatment regimen.

### VIII. Additional Considerations

The headers and subheaders between sections and subsections of this document are included solely for the purpose of improving readability and do not imply that features cannot be combined across sections and subsection. Accordingly, sections and subsections do not describe separate embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements (e.g., elements in block or schematic diagrams, elements in flow diagrams, etc.) without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

1. A method comprising:
receiving baseline data for a subject diagnosed with neovascular age-related macular degeneration (nAMD);
forming a plurality of predictor inputs for an outcome predictor using the baseline data and regimen data for a plurality of treatment regimens, wherein the plurality of predictor inputs comprises a different predictor input for each of the plurality of treatment regimens, wherein the outcome predictor comprises at least one machine learning model;
generating, via the outcome predictor, a plurality of treatment scores for the plurality of treatment regimens using the plurality of predictor inputs; and
selecting one of the plurality of treatment regimens as a selected treatment regimen for the subject based on the plurality of treatment scores.

2. The method of claim 1, wherein the selected treatment regimen maximizes vision improvement and minimizes an injection frequency.

3. The method of claim 1, wherein the selecting comprises:
determining whether a single treatment regimen of the plurality of treatment regimens or multiple treatment regimens of the plurality of treatment regimens meets a score criterion.

4. The method of claim 3, wherein the selecting further comprises:
responsive to the single treatment regimen meeting the score criterion, selecting the single treatment regimen as the selected treatment regimen for the subject; or
responsive to the multiple treatment regimens meeting the score criterion, identifying a treatment regimen of the multiple treatment regimens that meets a set of treatment burden criteria for the subject as the selected treatment regimen.

5. The method of claim 4, wherein the set of treatment burden criteria comprises at least one of a fewest number of injections, a lowest frequency of injections, a lowest dosage, a lowest drug strength, a reduced amount of monitoring, or reduced side effects.

6. The method of claim 1, wherein the generating comprises:
processing, via the outcome predictor, each of the plurality of predictor inputs independently to generate the plurality of treatment scores.

7. The method of claim 1, wherein:
the outcome predictor comprises a plurality of predictor models;
each of the plurality of predictor models comprises at least one machine learning model; and
each of the plurality of predictor models is configured to generate a treatment score of the plurality of treatment scores for a corresponding treatment regimen of the plurality of treatment regimens using a predictor input of the plurality of predictor inputs that corresponds to the treatment regimen.

8. The method of claim 1, wherein the baseline data comprises at least one of:
optical coherence tomography (OCT) image data;
clinical data that includes at least one of a visual acuity measurement, a central subfield thickness, a low-luminance deficit, age, or sex; or
retinal feature data extracted from segmented image data that has been generated from OCT image data corresponding to a baseline point in time.

9. The method of claim 1, wherein the regimen data for a corresponding one of the plurality of treatment regimens identifies a treatment and at least one of an administration frequency, a dosage schedule, or a monitoring schedule for the treatment.

10. The method of claim 1, wherein each of the treatment scores is a predicted visual acuity measurement.

11. The method of claim 1, wherein the at least one machine learning model comprises at least one of a linear regression model, a random forest (RF) model, a Gradient Boosting Machine (GBM) model, an Extreme Gradient Boosting (XGBoost), or a Support Vector Machine (SVM) model.

12. A method comprising:
receiving clinical data and imaging data for a subject diagnosed with neovascular age-related macular degeneration (nAMD) for a baseline point in time;
generating retinal feature data using the imaging data, wherein the retinal feature data comprises at least one of a pathology-related feature, a layer-related volume feature, or a layer-related thickness;
generating, via an outcome predictor comprising at least one machine learning model, a plurality of treatment scores for a plurality of treatment regimens;
selecting one of the plurality of treatment regimens as a selected treatment regimen for the subject based on the plurality of treatment scores.

13. The method of claim 12, wherein the selecting comprises:
determining whether a single treatment regimen of the plurality of treatment regimens or multiple treatment regimens of the plurality of treatment regimens meets a score criterion.

14. The method of claim 13, wherein the selecting further comprises:
responsive to the single treatment regimen meeting the score criterion, selecting the single treatment regimen as the selected treatment regimen for the subject; or
responsive to the multiple treatment regimens meeting the score criterion, identifying a treatment regimen of the multiple treatment regimens that meets a set of treatment burden criteria for the subject as the selected treatment regimen.

15. The method of claim 12, wherein the clinical data comprises at least one of a visual acuity measurement, a central subfield thickness, a low-luminance deficit, age, or sex for the baseline point in time and wherein the imaging data comprises optical coherence tomography (OCT) image data.

16. The method of claim 12, wherein each of the treatment scores is a predicted visual acuity measurement.

17. The method of claim 12, wherein the at least one machine learning model comprises at least one of a linear regression model, a random forest (RF) model, a Gradient Boosting Machine (GBM) model, an Extreme Gradient Boosting (XGBoost), or a Support Vector Machine (SVM) model.

18. A system for predicting a selected treatment regimen for a subject diagnosed with neovascular age-related macular degeneration, the system comprising:
a memory containing machine readable medium comprising machine executable code; and
a processor coupled to the memory, the processor configured to execute the machine executable code to cause the processor to:
receive baseline data for a subject diagnosed with neovascular age-related macular degeneration (nAMD);
form a plurality of predictor inputs for an outcome predictor using the baseline data and regimen data for a plurality of treatment regimens,
wherein the plurality of predictor inputs comprises a different predictor input for each of the plurality of treatment regimens,
wherein the outcome predictor comprises at least one machine learning model;
generate, via the outcome predictor, a plurality of treatment scores for the plurality of treatment regimens using the plurality of predictor inputs; and
select one of the plurality of treatment regimens as a selected treatment regimen for the subject based on the plurality of treatment scores.

19. The system of claim 18, wherein the selected treatment regimen maximizes vision improvement and minimizes an injection frequency.

20. The system of claim 18, wherein the processor is further configured to execute the machine executable code to cause the processor to:
determine whether a single treatment regimen of the plurality of treatment regimens or multiple treatment regimens of the plurality of treatment regimens meets a score criterion; and
responsive to the single treatment regimen meeting the score criterion, select the single treatment regimen as the selected treatment regimen for the subject; or
responsive to the multiple treatment regimens meeting the score criterion, identify a treatment regimen of the multiple treatment regimens that meets a set of treatment burden criteria for the subject as the selected treatment regimen.
